(19) United States
(12) Patent Application Publication
(10) Pub. No.: US 2018/0271641 A1
(43) Pub. Date: Sep. 27, 2018

(54) SURGICAL FASTENING DEVICE

(71) Applicant: Worcester Polytechnic Institute, Worcester, MA (US)

(72) Inventors: Sakthikumar Ambady, Shrewsbury, MA (US); Alexandra Czamara, Auburn, MA (US); Anne McLoughlin, Worcester, MA (US); Kyra McNamara, Chelmsford, MA (US); Lindsay Schneider, Niantic, CT (US); Emily Viloudaki, Madison, CT (US); Kristin Gallagher, Coventry, RI (US); Gabriela Meza, Wilmington, CA (US); Nicholas Brocato, Wyoming, RI (US); Robert J. Meislin, New York, NY (US)

(21) Appl. No.: 15/897,428

(22) Filed: Feb. 15, 2018

Related U.S. Application Data
(60) Provisional application No. 62/459,202, filed on Feb. 15, 2017.

(51) Int. Cl. A61F 2/08; B33Y 80/00

(52) U.S. Cl. CPC A61F 2/0811; B33Y 80/00; A61F 2002/0835; A61F 2002/087; A61F 2210/0004

(57) ABSTRACT

A biocompatible, biodegradable surgical fastening device employs opposed engagement surfaces having receptacles and protrusions, in which the receptacles are responsive to the protrusions on the opposed engagement surface. The fastening device has a generally planar construction defining the engagement surface, such that each engagement surface has a proximate end for engaging the opposed engagement surface, and a distal end for attachment to an anatomical structure. The distal end of two devices each connect to anatomical structures to be joined, and the engagement surfaces joined to each other at the proximate end to complete the repair. The distal end of the engagement surface defines a flexible attachment portion having a lateral attachment width, the lateral width configured for securement to the anatomical structure across a nonlinear disposition of the lateral width. The flexible lateral width conforms around an irregular shape of an anatomical structure such as a tendon, ligament or bone.

### RELATED APPLICATIONS

This application claims the benefit under 35 U.S.C. § 119(e) of U.S. Provisional Patent Application No. 62/459,202 filed Feb. 15, 2017, entitled “SURGICAL FASTENING DEVICE,” incorporated herein by reference in entirety.

### BACKGROUND

Surgical attachments are commonly used for repairing, supplementing and/or replacing connective tissue members such as tendons, ligaments and cartilage. The surgical attachments secure the connective tissue to skeletal members and other connective tissue in response to trauma, tears and degradation of the natural anatomical members. Sutures and bone screws or anchors are often employed for securing the connective tissue to structural elements including bone and cartilage.

### SUMMARY

A biocompatible, biodegradable surgical fastening device employs opposed engagement surfaces having receptacles and protrusions, in which the receptacles are responsive to the protrusions on the opposed engagement surface. The fastening device has a generally planar construction defining the engagement surface, such that each engagement surface has a proximate end for engaging the opposed engagement surface, and a distal end for attachment to an anatomical structure. The distal end of two devices each connect to anatomical structures to be joined, and the engagement surfaces join to each other at the proximate end to complete the repair. The distal end of the engagement surface defines a flexible attachment portion having a lateral attachment width, such that the lateral attachment width is configured for securement to the anatomical structure across a nonlinear disposition of the lateral width. The flexible nature of the device allows the lateral width to conform around an irregular shape of an anatomical structure such as a tendon, ligament or bone. The fastening device and the opposed engagement surfaces are defined by planar biocompatible materials.

Configurations herein are based, in part, on the observation that surgical attachments are employed to repair compromised anatomical structures by securing and/or reattaching connective tissue to corresponding natural members for effecting the repair. Unfortunately, conventional approaches to surgical attachment suffer from the shortcoming that anchors, screws and sutures tend to concentrate point forces, leading to tears and failure of the surgical repair. Accordingly, configurations herein substantially overcome the shortcomings of conventional anchors by providing a planar fastening device for securing a connective anatomical member along an entire lateral attachment width. The lateral attachment width extends across the planar surface for distributing forces imposed by the surgical attachment. The 2 dimensional, or planar, attachment has two portions joined to each other by a series of protrusions and receptacles, and is secured to anatomical members (bones, ligaments, tendons) across the lateral attachment width with glued and/or barbed surfaces. Sutures or tethers may also be employed across the attachment width. The planar attachment is constructed of biocompatible materials adapted for extruded or additive manufacturing, facilitating the use of 3D printers.

The disclosed approach is particularly beneficial in rotator cuff procedures. With an increasing need for rotator cuff surgeries to be performed on a variety of patient demographics, an adaption to current surgery technique is advised, as sutures commonly pull through the healing tendon, often leading to a secondary repair surgery. If this second surgery is conducted, the range of motion in the shoulder joint becomes limited and the rehabilitation process is extended, as well as causing increases to cost and recovery time. Configurations herein present a biocompatible surgical fastener to reattach the affected tendon into its insertion point with compatible mechanical properties that will work cohesively with the current surgical procedures.

In further detail, the surgical fastening device disclosed herein includes opposed engagement surfaces having receptacles and protrusions; such that the receptacles are responsive to engage the protrusions on the opposed engagement surface. Each engagement surface has a proximate end for engaging the opposed engagement surface and a distal end for attachment to an anatomical structure such as tendons, ligaments, bone and muscle. The distal end definies a flexible attachment portion having a lateral attachment width at the distal end, in contrast to conventional point load attachments such as sutures and anchors. The lateral attachment width is thus configured for securement to the anatomical structure across a nonlinear disposition of the lateral width. The opposed engagement surfaces are defined by planar biocompatible materials to facilitate natural tissue regrowth,

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1A is a side cutaway view of a surgical attachment device as disclosed herein;

FIG. 1B is a plan view of the surgical attachment device of FIG. 1A;

FIG. 2 is a perspective view of a pedestal and cone protrusion and receptacle in a surgical attachment device as in FIGS. 1A and 1B;

FIG. 3 is a perspective view of a pedestal and pedestal spacing defined receptacle;

FIGS. 4A and 4B are a perspective view of a protrusion and constrained width (keyhole) receptacle of the surgical attachment as in FIGS. 1A and 1B;

FIGS. 5A-5C are perspective, side elevation and plan views of an anatomical attachment of the surgical attachment devices of FIGS. 1-4B; and

FIGS. 6A-6B show an opposed clamp arrangement of anatomical attachment.

### DETAILED DESCRIPTION

Configurations below depict example arrangements of the disclosed approach. The surgical attachment device includes a generally flexible, resilient, planar attachment adapted for fabrication on 3D (3 dimensional) printers. Approaches depicted below include non-limiting examples and configurations, however other arrangements may be apparent.

Conventional approaches to surgical attachments include bone screws, bone anchors, sutures and hook-and-loop style fasteners sometimes referred to as bio-VELCRO®. One particular approach is disclosed in U.S. Pat. No. 6,039,741, which shows a hook-and-loop style of attachment. Many of these conventional approaches have fabrication issues on 3D printers, as finer structures and fibrous elements are difficult to fabricate on 3D printers. Many conventional approaches employ titanium and non-degradable polymers which do not promote natural tissue regrowth and healing.

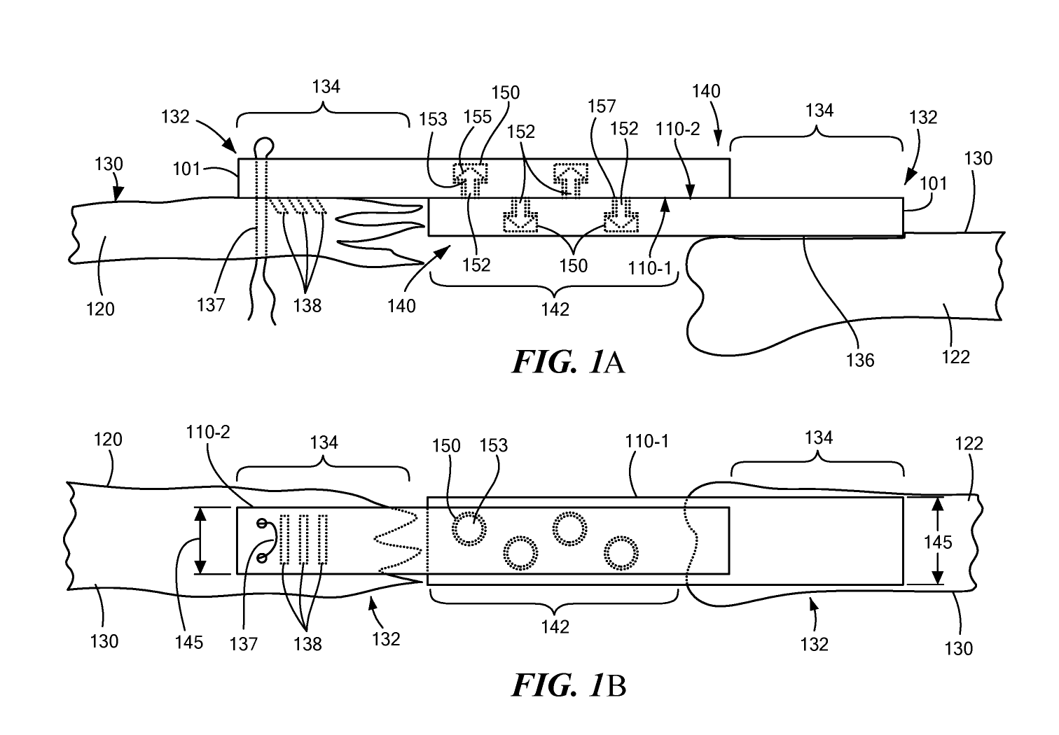

FIGS. 1A and 1B is a side cutaway view of a surgical attachment device 101 as disclosed herein. The surgical attachment device 101 has a generally planar shape, and is composed of flexible materials adapted for extruded construction, such as with a 3D printer. The general structure 100 depicted in FIGS. 1a and 1b includes opposed engagement surfaces 110-1, 110-2 (110 generally) on each surgical attachment device 101. Each engagement surface 110 is also configured to attach to an anatomical structure such as a tendon, ligament 120 or bone 122. The engagement surfaces 110 define a two-part approach to joining anatomical surfaces. Each engagement surface 110 has a proximate end 140 for engaging the opposed engagement surface 110 and a distal end 132 for attachment to an anatomical structure. Engagement surfaces 110 are first secured to an anatomical structure, using one or more of angled protrusions, adhesive, suture or surgical anchors. The engagement surfaces 110 are then drawn together in an opposed arrangement. The engagement surfaces 110 have receptacles 150 and protrusions 152, such that the receptacles 150 are responsive to the protrusions 152 on the opposed engagement surface 110. Therefore, each engagement surface 110 attaches to an anatomical surface 130 at a distal end 132 defining an anatomical attachment region 134, and attaches to the opposed engagement surface 110 at a proximate end 140 defining a surgical attachment region 142.

Referring to FIG. 1b, the distal end 132 defines a flexible attachment portion having a lateral attachment width 145, such that the lateral attachment width 145 is configured for securement to the anatomical structure across a nonlinear disposition of the lateral attachment width 145. The flexible, planar nature of the device 101 allows a contoured wrapping attachment to the anatomical structure 120, 122 using sutures, adhesive, or a barbed or tabbed structure, discussed further below. The devices 101 are defined by planar biocompatible materials responsive to 3D (3 dimensional) or additive manufacturing using an extruded medium via a motorized deposition carriage.

Referring to both FIGS. 1a and 1b, in a general configuration, the anatomical attachment region 134 is secured to the anatomical surface 130 by one or more of adhesive 136, sutures 137 or angled retentions 138 (e.g. barbs), discussed further below. The adhesive 136, sutures 137 or retentions 138 are distributed along the lateral attachment width 145 to avoid point loads and tearing which can occur with conventional approaches such as bone anchors or screws.

The surgical attachment device 101 is employed in a surgical procedure by attaching a plurality of opposed engagement surfaces to anatomical structures at the distal end 132, such that the distal end 132 defines a flexible attachment portion having a lateral attachment width 145. The lateral attachment width 145 is configured for securement to the anatomical structure across a nonlinear disposition of the lateral width for following contours and curves of the natural anatomical structure. Following attachment of the device 101 to both anatomical structures 120, 122, the opposed engagement surfaces 110 into are drawn into adjacency to close the surgical repair. Each of the opposed engagement surfaces 110 has at least one of receptacles 150 and protrusions 152 at the proximate end 140, such that the receptacles are responsive to the protrusions on the opposed engagement surface. The repair is secured through engaging the opposed engagement surfaces 110 by inserting the protrusions 152 into a corresponding receptacle 150 on the opposed engagement surface, in which each of the opposed engagement surfaces 110 is defined by planar biocompatible materials for facilitating healing and integration of natural anatomical tissue.

Depicted below are particular configurations of the receptacles 150, protrusions 152, and anatomical attachment regions. Various combinations of the receptacles and protrusions may occur in the surgical attachment region 142. The device 101 is formed from resilient materials that allow compressive deformation for insertion of the protrusions 152 into the receptacles 150. The receptacles 150 then retain the protrusions 152 through an interference fit or frictional retention. The anatomical attachment region 134 secures the device 101 to an anatomical structure such as bone, ligaments, tendons or cartilage from angled retentions 138, sutures 137 or adhesive 136. Adhesive 136 may be a biocompatible and/or moisture resistant formulation such as cyanoacrylate. Cyanoacrylate is a strong, biodegradable tissue adhesive that polymerizes upon contact with tissues. It can be used as a hemostatic agent or to “glue” tissues together in a surgical wound. Its binding is not affected by moisture or blood.

Biocompatibility materials used for device fabrication include polylactic acid (PLA), which is one of the more common commercially available biomaterials and has been widely approved by the FDA for many procedures. PLA is classified as a synthetic semi-crystalline material. Another biocompatible medium is PolyJet Photopolymer (MED610). The printed final product is colorless, transparent, and rigid. Biocompatibility tests in accordance with ISO 10993-1:2009 have commenced, however widespread FDA approval has not yet been achieved.

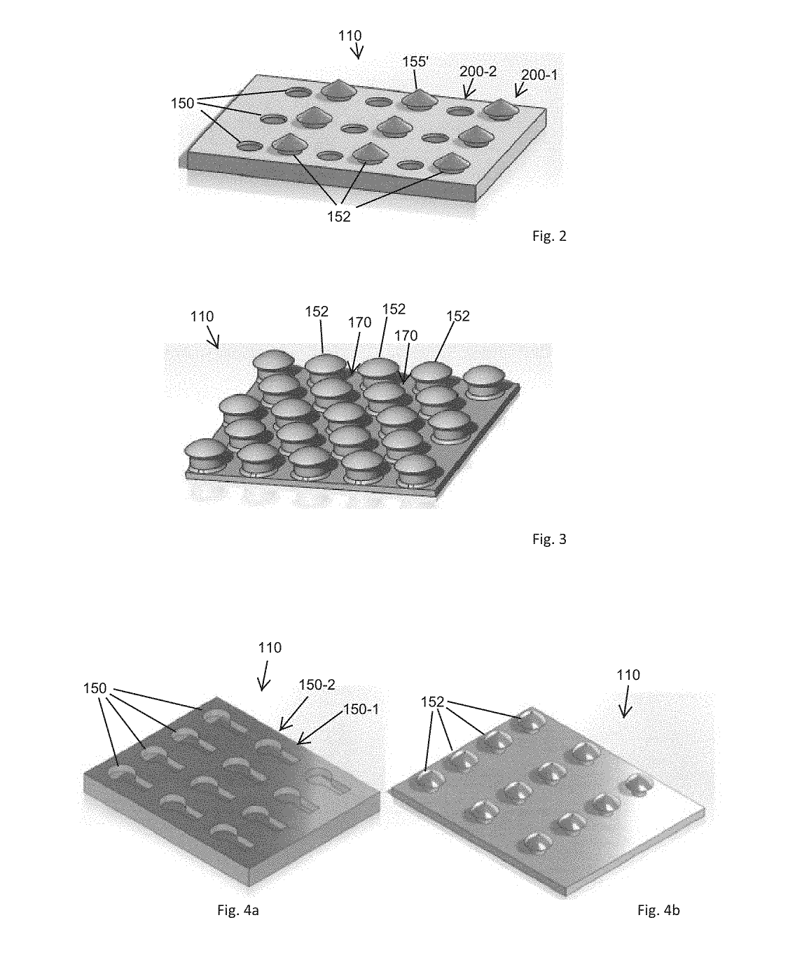

FIG. 2 is a perspective view of a pedestal and cone protrusion and receptacle in a surgical attachment device 101 as in FIGS. 1A and 1B. Referring to 1FIGS. 1A, 1B and 2, the opposed engagement surfaces 110 include a plurality of protrusions 152 and receptacles 150, such that the protrusions 152 and receptacles 150 are interleaved for engaging a corresponding protrusion 152 and receptacle 150 on the opposed engagement surface 110. The protrusions and receptacles are arranged in rows 200-1 of protrusions and receptacles 200-2. The interleaved arrangement is in inverse correspondence to the protrusions 152 and receptacles 150 on the opposed engagement surface 110 such that each protrusion 152 aligns with a corresponding receptacle 150. Each protrusion 152 includes a pedestal 153 extending substantially normal to the opposed engagement surface 110, and a fixation portion 155 having a larger area of cross section than the pedestal 153.

In the configuration of FIG. 2, The pedestal 153 extends from the surface 110, and the fixation portion 155 is defined by a cone 155′ on the pedestal 153 having a base wider than the pedestal 153 and a point extending distal from the surface. The receptacles 150 include apertures smaller than the base of the cone 155′ and at least as wide as the pedestal 153. When the device 101 lays flat, the fixation portion 155 has a cone or “mushroom” appearance on top of the pedestal, as the pedestal 153 is disposed between the engagement surface 110 and the fixation portion 155. The receptacle 150 has a smaller opening 157 widening to a larger area or diameter, such that the fixation portion 153 can deformably fit through the opening 157 into the receptacle but retains a larger area or diameter in the undeformed state for retention in the receptacle 150.

FIG. 3 is a perspective view of a pedestal and pedestal spacing defined receptacle. Referring to FIGS. 1a-3, a plurality of mushroom-shaped protrusions 152 are defined on each of the opposed engagement surfaces 110. A plurality of similarly shaped protrusions occupy each of the opposed engagement surfaces, and are spaced such that an area 170 between the fixation portions 155 is smaller than the fixation portions 155. An area between the pedestals 153 is at least as wide as the fixation portions 155. The fixation portions 155 are therefore adapted to engage a receptacle defined by a region or area 170 between a plurality of pedestals 153 on the opposed engagement surface, such that the region adapted to enclose the fixation portion 155 from an interference fit around the pedestal 153 as the fixation portions 155, once inserted, are blocked from withdrawal or removal by the fixation portions on the opposed surface 110. The “cone” shape of FIG. 2 and the “mushroom” shape of FIG. 3 may be interchanged and modified to employ a fixation portion 155 that is tapered and sized to deform or “snap” into the receptacle and has a cross section area slightly larger than the receptacle 150 for a secured engagement. A circular shape to the fixation portion allows the interference tolerances to be defined with respect to diameters of the respective protrusions and receptacles, however any suitable shape having deformation and area/size proportions for providing the frictional or interference fit will suffice. FIGS. 4A and 4B are a perspective view of the device 101 employing a protrusion and constrained width (keyhole) receptacle 150 of the surgical attachment as in FIGS. 1A and 1B. Referring to FIGS. 1a, 1b and 4A-4B, the opposed engagement surfaces 110 include a plurality of protrusions 152 on one of the engagement surfaces 110 and a plurality of receptacles 150 on the opposed engagement surface 110. The receptacles 150 are elongated and having portions of varying width to define apertures including a retention portion 150-1 having a width smaller than the fixation portion 155 and at least as wide as a shortest cross section of the pedestal 153. The apertures also include an insertion portion 150-2 adapted for insertion of the fixation portion 155. The insertion portion 150-2 has a larger width or area for protrusion 152 insertion as above, and the engagement surface 110 is disposed towards the retention portion 150-1 to secure the fixation portion 155 beneath or behind the narrower width of the retention portion 150-1.

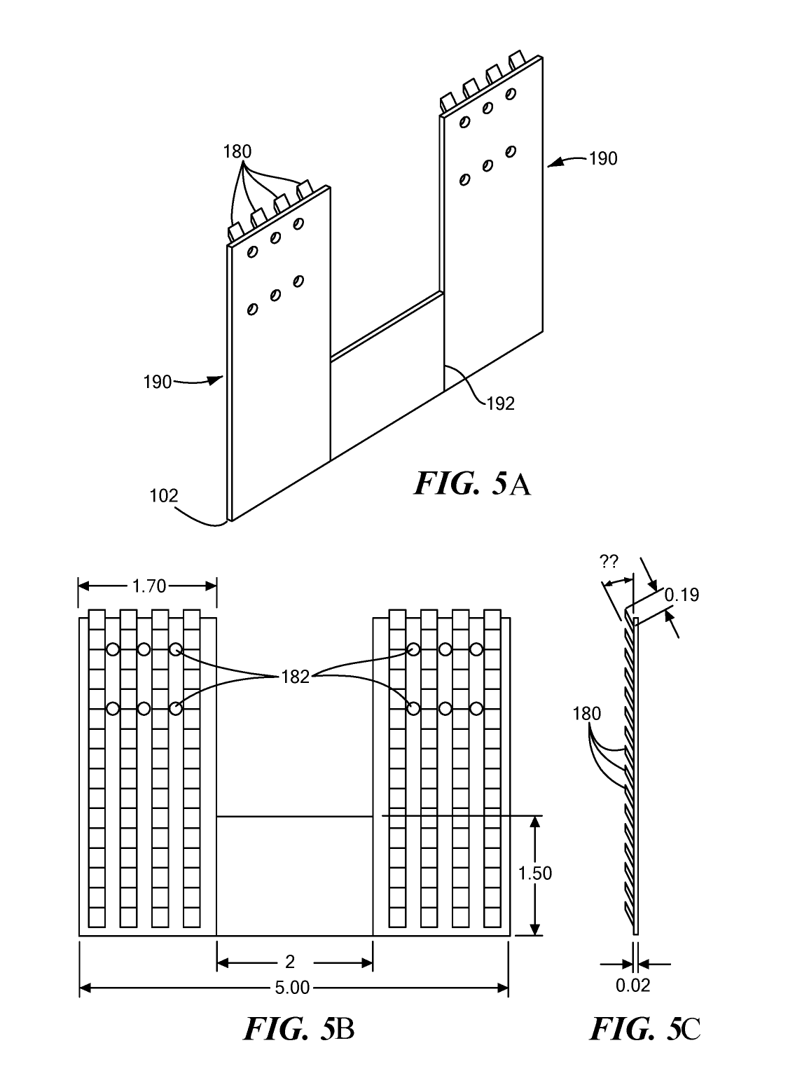

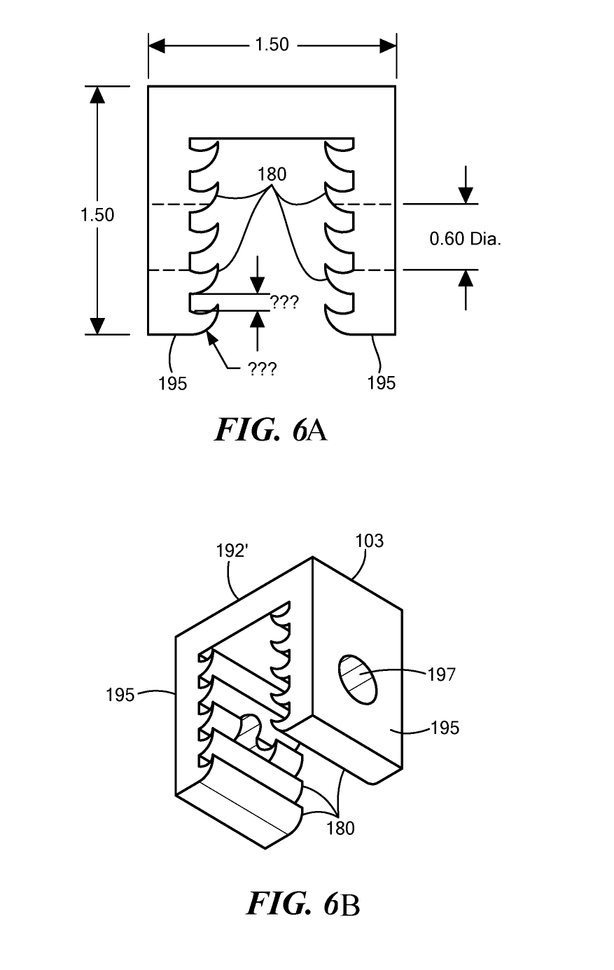

The configurations of FIGS. 2-4B disclose engagement mechanisms of the surgical attachment region 142 for securing the opposed attachment surfaces 110. The distal end 132 of each attachment device 101 is secured to an anatomical structure or feature to complete the surgical repair, now discussed with respect to FIGS. 5A-6B.

FIGS. 5A-5C are perspective, side elevation and plan views of an anatomical attachment of the surgical attachment devices 101 of FIGS. 1-4B. The approach of FIGS. 5A-5C is operable as a stand-alone attachment to conventional suture or anchor approaches, in addition to providing an attachment for the distal end 132. Referring to

FIGS. 1a, 1b and 5A-5C, angled retentions 138 (e.g. barbs) disposed on the anatomical tissue side of the distal end 132 provide a cleated attachment to anatomical structures (e.g. tendons, ligaments, muscle and bone). The angled retentions 138 may have a width suitable for insertion into flexible anatomical structures such as ligaments 120 and tendons, or for rigid elements such as bone 122. A narrower or “barb” point may be more suited for bone engagement, and wider “tabs” 180 may be effective for softer structures to avoid tearing. In either case, sutures 137 or adhesive 136 may supplement. The biocompatible construction of the device 101 allows natural healing and integration of the attached structures over time, in contrast to conventional “bio Velcro”, screws or anchors which remain indefinitely.

In an alternate arrangement, a U-shaped attachment structure 102 of FIG. 5A serves as a foldable attachment which brings barbed surfaces 190 into an opposed arrangement around an anatomical member by folding along a hinge 192. 7. A plurality of angled protrusions 180 are disposed on the flexible attachment portion, and the angled protrusions or retentions 138 are adapted for cleated engagement with anatomical members. Rows 182 of suture holes permit sutures 137 along a lateral width as in FIGS. 1A and 1B for dispersing force, rather than focusing a point load onto a single suture.

FIGS. 6A-6B show an opposed clamp arrangement of anatomical attachment 103. A transverse member 192′ disposes the opposed surfaces 195 according to a width of the transverse member 192. The angled retentions 137 are defined by curved rows of cleat shaped tabs 180 extending continuously across the opposed surfaces 195. In the configurations of FIGS. 5A-6B, therefore, a plurality of barbs are disposed on the flexible attachment portion and angled toward the direction of forces on the attachment portion. An anchor hole 197 may also be employed for insertion of a fixation screw or anchor through the anatomical member for greater attachment strength.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A surgical fastening device, comprising:
opposed engagement surfaces having receptacles and protrusions; the receptacles responsive to the protrusions on the opposed engagement surface, each engagement surface having a proximate end for engaging the opposed engagement surface and a distal end for attachment to an anatomical structure; and
the distal end defining a flexible attachment portion having a lateral attachment width at the distal end, the lateral attachment width configured for securement to the anatomical structure across a nonlinear disposition of the lateral width;
the opposed engagement surfaces defined by planar biocompatible materials.

2. The device of claim 1 wherein the opposed engagement surfaces include a plurality of protrusions and receptacles, the protrusions and receptacles interleaved for engaging a corresponding protrusion and receptacle on the opposed engagement surface.

3. The device of claim 1 further comprising a plurality of protrusions on one of the engagement surfaces and a plurality of receptacles on the opposed engagement surface.

4. The device of claim 1 wherein the protrusions further comprise:
a pedestal extending substantially normal to the opposed engagement surface;
a fixation portion having a larger area of cross section than the pedestal, the pedestal disposed between the engagement surface and the fixation portion.

5. The device of claim 4 further comprising:
a plurality of similarly shaped protrusions on each of the opposed engagement surfaces,
wherein the fixation portions are adapted to engage a receptacle defined by a region between a plurality of pedestals on the opposed engagement surface, the region adapted to enclose the fixation portion from an interference fit around the pedestal.

6. The device of claim 1 wherein the protrusions include:
a pedestal extending from the surface;
a cone on the pedestal having a base wider than the pedestal and a point extending distal from the surface; and
the receptacles including apertures smaller than the base of the cone and at least as wide as the pedestal.

7. The device of claim 1 further comprising a plurality of angled protrusions on the flexible attachment portion, the angled protrusions adapted for cleated engagement with anatomical members.

8. The device of claim 1 further comprising a plurality of barbs disposed on the flexible attachment portion, the barbs angled toward the direction of forces on the attachment portion.

9. The device of claim 4 wherein the apertures include a portion having a width smaller than the fixation portion and at least as wide as a shortest cross section of the pedestal.

10. The device of claim 9 wherein the apertures include an insertion portion adapted for insertion of the fixation portion.

11. A method of installing surgical fastening device, comprising:
attaching a plurality of opposed engagement surfaces to anatomical structures at a distal end, the distal end defining a flexible attachment portion having a lateral attachment width, the lateral attachment width configured for securement to the anatomical structure across a nonlinear disposition of the lateral width;
drawing the opposed engagement surfaces into adjacency, each of the opposed engagement surfaces having at least one of receptacles and protrusions at a proximate end; the receptacles responsive to the protrusions on the opposed engagement surface,
engaging the opposed engagement surfaces by inserting the protrusions into a corresponding receptacle on the opposed engagement surface;
the opposed engagement surfaces defined by planar biocompatible materials.

12. The method of claim 11 wherein the opposed engagement surfaces include a plurality of protrusions and receptacles, the protrusions and receptacles interleaved for engaging a corresponding protrusion and receptacle on the opposed engagement surface.

13. The method of claim 11 further comprising engaging a plurality of protrusions on one of the engagement surfaces and a plurality of receptacles on the opposed engagement surface.

14. The method of claim 11 wherein the protrusions further comprise:
a pedestal extending substantially normal to the opposed engagement surface;
a fixation portion having a larger area of cross section than the pedestal, the pedestal disposed between the engagement surface and the fixation portion.

15. The method of claim 14 further comprising:
engaging a plurality of similarly shaped protrusions on each of the opposed engagement surfaces,
wherein the fixation portions are adapted to engage a receptacle defined by a region between a plurality of pedestals on the opposed engagement surface, the region adapted to enclose the fixation portion from an interference fit around the pedestal.

16. The method of claim 11 wherein the protrusions include:
a pedestal extending from the surface;
a cone on the pedestal having a base wider than the pedestal and a point extending distal from the surface; and
the receptacles including apertures smaller than the base of the cone and at least as wide as the pedestal.

17. The method of claim 11 further comprising engaging a plurality of angled protrusions on the flexible attachment portion in a cleated engagement with anatomical members.

18. The method of claim 17 wherein the angled protrusions include a plurality of barbs disposed on the flexible attachment portion, the barbs angled toward the direction of forces on the attachment portion.
